(19) United States
(12) Patent Application Publication
(10) Pub. No.: US 2009/0170203 A1
(43) Pub. Date: Jul. 2, 2009

(54) METHODS FOR FEMALE MAMMALIAN SPERMATOGENESIS AND MALE MAMMALIAN OOGENESIS USING SYNTHETIC NANOBIOLOGY

(76) Inventor: Gregory P. Aharonian, San Francisco, CA (US)

Correspondence Address: DAVID P. LENTINI, 53 Clark Road, North Berwick, ME, 03906-6310, US

(21) Appl. No.: 11/968,180

(22) Filed: Jan. 1, 2008

(51) Int. Cl. C12N 15/87; C12N 5/06

(52) U.S. Cl. 435/455; 435/366

(57) ABSTRACT

Herein are disclosed methods for producing female sperm by incorporating a female's chromosomes into a sperm cell via mostly natural spermatogenesis processes. The methods include transplanting (altered) female stem cells or (altered) cloned germ cells into sterilized testes of a male. Diploid female stem/germ cells are altered in two ways—transdifferentiation to facilitate the expression of spermatogenesis factors (for example, using artificial chromosomes) and/or retrodifferentiation to increase pluripotency and imprinting erasures. The altered cells are transplanted into (artificial) male testes to develop into sperm, which are used to fertilize an egg of a second female, or an egg of the original female. Also disclosed herein are methods for producing male eggs by adding an extra X chromosome to an adult male's germ cells, and cultivating the germ cells in vitro.

### PRIORITY

This patent application claims priority to co-pending U.S. patent application Ser. No. 11/772,568, filed 2 Jul. 2007, titled “Methods for Female Mammalian Spermatogenesis”, the disclosure of which is incorporated herein by reference in its entirety and for all purposes.

### FIELD OF THE INVENTION

The present invention relates generally to methods for mammalian reproduction. More specifically, the present invention includes methods for developing sperm containing a female's chromosomes, or developing eggs containing a male's chromosomes, and the sperm or eggs so produced. More particularly, the present invention includes methods for transplanting genetically-altered female germ and stem cells into functional, but sterilized (artificial), male testes to produce female sperm; and methods for cultivating genetically-altered male germ and stem cells to produce functional male eggs. The invention has applications in the fields of genetics, medicine and animal husbandry.

### BACKGROUND OF THE INVENTION

A controversial social issue on the new millennium is that of same-sex marriage should two people of the same sex have the right to be married? Some countries have legalized same-sex marriage (Netherlands 2001, Belgium 2003, Canada 2005, Spain 2005, South Africa 2006). Some states in the United States have also legalized same-sex relationships (marriage: Massachusetts 2004; civil unions: Vermont 2000, Connecticut 2005, New Jersey 2006). Other states and countries have explicit or implicit bans on same-sex marriages, preferring only different-sex marriages. The main, if not only, objective reason for opposing same-sex marriage is that same sex procreation is impossible two men cannot conceive a child that has genetic material only from both men, and similarly for two women. This impossibility distinguishes same-sex marriage from interracial marriage, bans on which the U.S. Supreme Court ruled unconstitutional in the 1967 case Loving v. Virginia. But is same sex procreation impossible, so that same-sex couples are forever denied equal protection under marriage laws? Or like brain surgery, face transplants and other “unnatural” medical technologies, are there clinical techniques to achieve same-sex procreation?

Same-sex procreation requires that the properly imprinted haploid chromosomes (including the X chromosome) of a male be carried in a fertilizable egg (to be fertilized by a second male's sperm), or the properly imprinted haploid chromosomes of a female be carried in the body of a viable sperm (to fertilize the egg of a second female), processes that can be described by the phrase “transsexual gametogenesis”. (The term “gametogenesis” as used herein refers in general to spermatogenesis and oogenesis.) Experiments have ruled out the possibility of creating female sperm by directly injecting female chromosomes into an empty sperm nucleus: the injected chromosomes are not paternally imprinted; and the injected sperm DNA is not sufficiently condensed (i.e. sperm DNA is packaged in a special way during spermatogenesis) to be viable. Some of the methods disclosed herein are directed towards generating sperm or spermatids (spermatogenesis) carrying a male-imprinted haploid chromosome set derived from a female individual, thereby producing “female sperm”. Germ line cellular transformations (e.g., using artificial SRY-negative Y chromosomes) are also disclosed to allow such procedures for making female sperm under conditions as naturally as possible (i.e., under the control of existing biological processes). Other methods disclosed herein provide “male eggs” (oogenesis).

### Natural Production of Sperm in Males

Producing female sperm requires re-engineering processes for production of sperm in males by compensating for differences in genotype and imprinting between males and females. The production of sperm in males comprises the following stages:
Zygote imprinting erasure begins
Diploid germ cell creation in the embryonic yolk sac
Germ cell migration to the genital ridge
Mitotic germ cell divisions to form spermatogonia; male imprinting begins Suspension of mitotic divisions until puberty
Spermatogenesis: mitotic divisions and imprinting of spermatogonia into spermatocytes
Spermatogenesis: meiotic divisions and imprinting of spermatocytes into spermatid
Spermiogenesis: transformation of tailless spermatids into sperm

Primordial germ cells (PGCs) are first recognizable in the yolk sac a few weeks after fertilization. About four weeks after fertilization, the PGCs migrate towards the genital ridge where they continue to undergo a period of growth and proliferation. The medulla of the genital ridge eventually gives rise to the seminiferous cords, which lengthen and become extensively folded to form the seminiferous tubules.

Migratory primitive germ cells that have reached these medullary cords, termed gonocytes, then move to a position adjacent to the basal lamina of the cords and enter a nonproliferative growth phase. By about 10 weeks after fertilization, the gonocytes mitotically differentiate to form primitive spermatogonia, which are progenitors to the eventual sperm.

In the male testis, spermatogenesis and spermiogenesis occur as germ cells move through the testis and supporting cells in the testis needed to produce sperm, in particular Sertoli and Leydig cells. Sertoli cells have receptors for follicle stimulating hormone (FSH) and testosterone, important regulators of spermatogenesis. Hormonal interactions between these supporting cells, non-testis organs (hypothalamus and pituitary glands), and the male germ stem cells regulate the growth of sperm.

After puberty, for example in humans, males start producing sperm. It takes about ninety days from stem cell division of spermatogonia until viable sperm are produced. This process continues throughout the lifetime of the male, though decreasing in productivity as the body ages. However, testicle production can drop as low as one sperm, and still lead to fertilization, using a variety of microsurgical techniques such as intracytoplasmic sperm injection.

Many of these natural steps can now be achieved artificially cloning to acquire diploid germ cells, chemical erasing of imprinting markings, transplantation of germ cells into chemically castrated testes, and in vitro spermatogenesis. While such artificial medical techniques have been developed to help with men with dysfunctional spermatogenesis (often seen in men with defective genes on the Y-chromosome), heretofore no one has identified any combination of such techniques that could be useful for people with the ultimate in Y-chromosome dysfunctionality (having no Y-chromosome) women. Similarly, others have failed to identify medical techniques for people desiring eggs who have the ultimate in X-chromosome dysfunctionality (having one X-chromosome) men.

### Male-Female Gonad Similarities

At the earliest stages of embryo development, the first five to seven weeks or so, the gonads of XY and XX individuals do not exhibit detectable morphological differences and are essentially female. For example, during these stages both male and female embryos have a double set of sex ducts. In the male gonad, primordial germ cells in the cortex begin to degenerate while the testis cords in the center of the gonad continue to develop into seminiferous tubules. The gonad is thus converted into a testis. In the female gonad the central testis cords disappear, while the oocytes in the peripheral cortex survive and become surrounded by follicle cells; the gonad is developing into an ovary. Either event is triggered by the migration of primordial germ cells to the gonad.

Mammalian embryos are initially “sexless”, with the default pathway being ovarian differentiation (glibly, men from ovaries, not women from ribs). The presence of the SRY/TDF gene (sex-determining region Y/testis-determining factor gene) on the Y chromosome (for primates, for example) forces the gonad to become male. The DSS (dosage-sensitive sex reversal) locus also suggests being female as the default case for mammals, with SRY proposed to override one or more genes in DSS, and with two copies of DSS overwhelming SRY, causing male-to-female sex reversal [JIM98]. For males, epithelial somatic cells of the bipotent gonadal primordium differentiate into Sertoli cells, and this in turn triggers testis development [MCL91].

### Epigenetics/Imprinting

One major difference between male and female gametogenesis is imprinting. Imprinting is an epigenetic, gamete-of-origin-, and therefore parent-of-origin-,dependent modification of the genome, i.e., changes in DNA function without changes in DNA sequences. Typically, all humans have pairs of genes, one on each chromosome inherited from each parent, that are both expressed in cells. One class of imprinted genes are those that are parentally imprinted. When an autosomal gene at an imprintable location on a chromosome passes through gametogenesis of one sex, its ability to be expressed is unaffected. However, when this gene passes through gametogenesis of the opposite sex, it becomes inactivated it cannot be expressed. Such an inactivated gene is termed “imprinted”. Approximately one percent of human genes are imprinted. Some imprinted genes control fetal growth (such as the IGF2/H19 pair), as evidenced by gynogenetic embryos (no male imprints) having poor placental development but normal embryonic development, while androgenetic embryos (no female imprints) having the reverse. The results of parental imprinting have been long recognized by phenotypical effects, as far back as 3000 years by mule breeders in Asia Minor. Other epigenetic modifications include histone modifications and use of non-coding RNAs, but imprinting dominates. Epigenetics can be viewed as an evolutionary gift from our sexually liberal female placental ancestors, who survived better by always having generally healthy babies (averaged sized embryos) from multiple fathers (who survived better by having their children start out as larger embryos at the expense of imperiling future offspring from other fathers).

Only in recent years, however, have the genetic processes underlying imprinting been gradually brought to light, in particular, medical problems caused by faulty imprinting. For example, one of the major problems with nuclear-transfer cloning is that the cloned DNA is not properly imprinted, leading to cloned animals with abnormal phenotypes, assuming the cloned embryos survive at all to birth (only a low percentage so survive). In recent years, biologists have similarly noted that the imprinting problems that arise with cloning also arise in the creation of artificial sperm and eggs. In general, mastering epigenetic reengineering is the key to clinical acceptance of therapeutic uses of regenerated adult stem cells. Methods to compensate for transsexual gametogenesis problems due to differences in male and female imprinting and other epigenetic phenomenon are disclosed herein. A second class of imprinted genes involves the entire X chromosome in women, where early in development, one X chromosome in each cell of a woman is randomly imprinted (“X-inactivation”).

### Stem Cell Biology

Many cells live for only a short time and so must be replaced periodically. Since most cells lose their mitotic potential in the course of terminal differentiation, they are not able to create their replacements. Stem cells, however, possess two fundamental properties: the ability to self-renew and the ability to produce numerous differentiated progeny. Stem cells can be classified into two types according to how they self-renew: stereotypic and populational. Stereotypic stem cells self-renew strictly by asymmetric divisions that produce a daughter stem cell and a differentiated daughter cell. Populational stem cells divide symmetrically to produce two daughter cells each of which has an equal probability of differentiating. In mammals, it has yet to be determined which type of behavior germ stem cells exhibit, though germ cells are “ageless” as compared to somatic cells in that their chromosome ends are maintained by high levels of telomerase.

There are a variety of families of stem cells, including hematopoietic, mesenchymal, epithelial and germinal. The differentiation pathways for hematopoietic stem cells start with a pluripotent stem cell and ends with a variety of blood system cells such as red blood cells (erythrocytes), white blood cells (monocytes), and platelets. The technique of bone marrow transfer is based on the goal of transplanting hematopoietic stem cells from one animal to another (or in cases of chemotherapy, removing bone marrow, applying chemotherapy (which can damage bone marrow), and returning the bone marrow back after treatment).

For reproduction, there are at least two totipotent germ stem cells: the spermatogonia in male testis and the oogonia in the female ovaries, both of which are derived from primordial germ stem cells in the embryo.

The terminal differentiation pathways of oogonia and spermatogonia in mammals are similar until the final stages of terminal differentiation. Indeed the similarities are quite interesting. For example, some of the stages of meiotic division for sperm can take place if the spermatogonia are inserted into a female egg [SAS98].

### Female Same-Sex Procreation and Female Sperm

The possibility of offspring from two females has been speculated about in the past in a general way, but without much success in providing reliable methods for transsexual gametogenesis. (For example, a December 2007 search of the MEDLINE medical database PUBMED, using the phrases “female sperm”, “male egg”, and “same-sex procreation”, found no uses of these phrases. Such failure of others to use these phrases demonstrates a general lack of research by those skilled in the art with regards to the methods disclosed herein.) The speculations on the possibility of generating offspring from two females has followed two suggested pathways. The first pathway suggested included the production of chimeras, i.e., fusing two embryos (having either two mothers and two fathers, two mothers and one father, or two fathers and one mother—achieved in mice, suggested for human homosexual procreation in [SIL97]). However there are many problems with this procedure, including the use and discarding of multiple embryos, the physiological “patchiness” of resulting chimeras (think of Calico cats), the politics of abortion, etc. and the presence of multiple male chromosomes in a child with two mothers.

In 1968 researchers noted that non-mosaic Klinefelter men, i.e. men who have a 47, XXY genotype, somehow produce functional sperm. Surprisingly, Klinefelter researchers in the following forty years failed to publish any speculations that adding an artificial Y chromosome to a female diploid germ cell could create the men's parallel, a Klinefelter woman, whose germ cells could be transformed into sperm using the same, or similar, mitotic and meiotic pathways to those seen in Klinefelter men.

Later observations by McLaren noted that for pre-natal germ cells “the decision as to whether it is oogenesis or spermatogenesis on which they initially embark depends only on their environment, however, and not at all on their own chromosomes”, [MCL95]. Evans et al. twenty years earlier [EVA77] also observed that “ . . . the sex of a germ cell is not an autonomous property but is determined by the nature of the gonad in which it finds itself”. Indeed, in the 1980s, it was observed that male mice could have developed their own eggs by colonizing their developing adrenal glands with primordial germ cells. The resulting male eggs, except for their localization and for being surrounded by adrenal cells, were indistinguishable from quiescent oocytes in unilaminar ovarian follicles. Most likely such XY oocytes are incompetent for post-fertilization development in humans. For example, while an untreated female cell could start down the male gametogenesis pathway (or male cells down the female pathway), these observations failed to consider completion in light of missing or interfering gametogenesis genes. But both McLaren and the Evans group failed to consider using artificial Y chromosomes to make female germ cells more amenable to becoming sperm, and failed to consider how to deal with imprinting requirements with adult cells.

In the early 1990s, Brinster and Zimmerman developed techniques to correct genetic defects in the germ line of a male. Using their techniques [BRN99], stem cells are removed from a male's testes. After the male's testes are sterilized to kill off all remaining defective stem cells, the stem cells are reintroduced into the testes where they thrive and produce sperm. Such stem cells can be altered in vitro to correct gene defects before reintroduction into the testes. In 1992, Brinster and Zimmerman suggested transplanting female cells into the testes [BRN93], but failed to address the problems of which female cells to transplant, how to transform female imprinting patterns into male imprinting patterns (a problem which plagues cloning), and how to compensate for the missing Y chromosome. The latter failure is crucial since some Y-linked genes in spermatogenic cells are essential for the spermatogenesis process (such as the nuclear-expressed RNA-Binding Motif Y (RBMY) genes), a process which is disrupted where the female cells have no Y chromosome, a disruption unacceptable for any clinical/reproductive use of female sperm for humans that is subject to governmental regulations. Chuma and colleagues report [CHU05] report on injecting primordial germ cells using Brinster's technique.

In 1997, Japanese scientists mostly created female sperm in chickens by transplanting sex-reversed female WW primordial germ cells into chicken testes (female chicken cells are normally ZW heterogametic). But the Japanese were unable to create complete female chicken sperm. Notably, they failed to mention artificial chromosomes as a means to compensate for any missing genes. Moreover, they failed to mention the applicability of their techniques to human cells. Their paper fails to refer to Brinster's suggestion of this procedure in 1992 (“Differentiation of female chicken primordial germ cells into spermatozoa in male gonads”, Tagami et al., Develop. Growth Differ., 1997, vol 39, pages 267-71). Similarly, in 2001, West and Cibelli propose a method of cloning animals from adult animals, where the cloned offspring are of the opposite sex of the adult animal, by adding/deleting X and Y chromosomes from the cells of the adult animal before using nuclear transfer cloning techniques [WES02]. Their technique fails to be useful for humans because of the imprinting problems associated with cloning, and the unacceptable ethical problems of creating a new human who decades later can supply sperm or eggs to his/her even older “parent”.

Starting in 2003, scientists reported successes with converting mouse embryonic stem cells in vitro part, or all, of the way to sperm and eggs, in some cases achieving fertilization. Some research focused on converting male cells to sperm, and female cells to eggs, but failed to mention how to achieve cross-sex conversions and the additional problems that arise such as compensating for missing gametogenesis genes or switching imprinting patterns, and they failed to research the use of testicular transplantation to achieve spermatogenesis more naturally. Other researchers explored how mouse embryonic stem cells of either sex could start the process of becoming both sperm and eggs, but also failed to report results on compensating for mission gametogenesis genes or switching imprinting patterns, and they also failed to research the use of testicular transplantation [SCH03],[SCH05],[SCH06]. In parallel, other researchers explored the same possibility for using human embryonic stem cells [AFL05],[AFL06], though again in both cases, they failed to specify how to compensate for missing or interfering gametogenesis gene (e.g., using artificial chromosomes), and failed to specify how to use in vitro environments to complete gametogenesis.

In 2006, Karim Nayernia and scientists in Germany reported on creating male sperm from embryonic stem cells and the possibility of creating sperm from bone marrow stem cells [NAY06]. While Nayernia's publications failed to discuss the possibility of using these techniques to create female sperm, a few news stories raised the question of such a possibility. Both Nayernia's publications, and the news stories, failed to discuss how to solve problems due to imprinting when using female cells. They also failed to discuss the need to compensate for missing Y chromosome genes in female XX cells and the benefits, e.g., of using artificial Y chromosomes to so compensate for genes such as RBMY. A February 2006 paper by Nayernia and colleagues also failed to discuss these issues [NAY07]. Five years earlier, newswires reported a failure of other researchers to apply an earlier male mouse sperm creation technique to creation of human female sperm [BIR02]. One reason for such failures, given partial successes with animals such as mice, is that cellular techniques do not always obviously transfer across animal types. For example, mouse and human male germ cell erasure and establishment have different timing patterns; RBMY binds differently than its homologues in mice and humans; genes (such as IGF2R) are imprinted differently in mice and humans. As Gokhale and Andrews note in 2006 [GOK06], “Not surprisingly, as a more detailed molecular understanding of stem cells is gleaned it has become apparent that there are substantial differences between apparently equivalent mouse and human cells, . . . . Meanwhile, given the many examples of species differences, we must conclude that the extrapolation of conclusions from one species to another must be undertaken with caution.”

All published research on manipulating sperm production processes has failed to consider the problem of developing techniques to address fundamental barriers to generate female sperm, for example, techniques for addressing missing Y-chromosome effects, adjusting parental imprinting [THO05], X inactivation, and female germ cell—Sertoli cell interactions (and similarly for male eggs). Additionally, all published research on manipulating sperm production processes has failed to consider the use of adult female diploid germ cells (e.g, found in the ovarian surface epithelium). Indeed, an ethics article in 2005 [TES05] argued that making female sperm is more difficult than making male eggs, because of the complexities of compensating for the Y chromosome, a compensation for which the article claimed there are no obvious solutions. The failure to address these problems makes it impossible to prepare female sperm cells or male eggs suitable for use in fertilization for human beings. Moreover, a variety of recent articles have emphasized the problems and dangers of producing artificial sperm and eggs where imprinting and Y chromosome effects are not fully compensated for (e.g., by transplanting altered female germ cells into a male testes, and/or altering the female cells with artificial chromosomes carrying spermatogenesis genes): “In vitro spermatogenesis as a method to bypass pre-meiotic or post-meiotic barriers blocking the spermatogenesis process: genetic and epigenetic implications in assisted reproductive technology” by Georgiou et al., Andrologia, October 2007, vol. 39, no. 5, 159-176; “Artificial sperm and epigenetic reprogramming” by Diana Lucifero and Wolk Reik, Nature Biotechnology, vol. 24, no. 9, September 2006, 1097-1098; “Epigenetic decisions in mammalian germ cells” by Christopher Schaefer et al., Science, 20 Apr. 2007, vol. 316, 398-399; “Gametes from embryonic stem cells: a cup half empty or half full?” by George Daley, Science, 20 Apr. 2007, vol. 316, 409-410; and “Do cloned mammals skip a reprogramming step” by Josef Fulka Jr. et al., Nature Biotechnology, vol. 22, no. 1, January 2004, 25-6. With work on lower animals such as mice and chicken, there is little concern about the health of offspring produced using artificial sperm, so there was no motivation to address such compensation problems for humans.

Similarly, over the years, biologists have reported on the fertility of sex-reversed XY female mammals (such as mice, fish, and horses) and on the heightened fertility of 47,XXY female mammals (such as mice and humans), while failing to discuss the possibility of mammalian male eggs produced from the germ cells of 47,XXY men or 47,XY men whose germ cells have added an extra X chromosome.

What is needed to fully enable the production of female sperm in humans are methods that include the expression of all of the required genes for spermatogenesis, and that allow male imprinting to be achieved for female cells as naturally as possible, preferably by using in vivo environments as much as possible. Some of the methods provided by the present invention enable the procreation of offspring that are solely the genetic product of two women. Similarly, what is needed to fully enable the production of male eggs in humans are methods that transform adult male XY germ cells into XXY germ cells and allow female imprinting to be achieved for the male cells as naturally as possible. Some of the methods provided by the present invention enable the procreation of offspring that are solely the genetic product of two men through the production of male eggs.

### SUMMARY OF THE INVENTION

The present invention provides methods for creating female sperm. In some embodiments, the methods of the invention include: creating female germ cells (e.g., via cloning and/or retrodifferentiating female stem cells to make them more pluripotent); expanding in vitro the number of such cells; transdifferentiating these cells to facilitate the expression of spermatogenesis factors (for example, using synthetic or artificial Y chromosomes); transplanting the resulting cells into a sterilized testes or artificial testicular environment; and allowing the transplanted cells to develop into competent spermatid nuclei or biologically functioning sperm. In one embodiment, a second female's egg is fertilized using these spermatids or sperm, or the first female's egg is fertilized with her own sperm for self-fertilization, an alternative to cloning by nuclear transfer.

The present invention, at a minimum, also provides methods for creating male eggs. In some embodiments, the methods of the invention include: transdifferentiating adult male germ cells to facilitate development into an egg (for example, adding an extra X chromosome); transplanting the resulting cells into an artificial ovarian environment; and allowing the transplanted cells to develop into competent eggs. In one embodiment, a second male's sperm is used to fertilize such male eggs, or the first male's egg is fertilized with his own sperm for self-fertilization, an alternative to cloning by nuclear transfer.

The features and advantages of the present invention will be more readily understood and apparent from the following detailed description of the invention, which should be read in conjunction with the accompanying drawings, and from the claims which are appended at the end of the detailed description.

### DETAILED DESCRIPTION

Reflecting the uncertainty of the ultrastructure and bioequivalence of stem cells, non-germ stem cells will be referred to as “stem cells”, with the appropriate modifier, such as “hematopoietic stem cells”. Primordial, primitive and differentiated germ cells such as oogonia and spermatogonia will be referred to as “germ cells” or “germinal cells”, which are often referred to in the medical literature somewhat confusingly as either “germ cells” or “stem cells”.

For the methods disclosed herein, the phrase “spermatogenesis” refers to the entire process of transforming germ cells into sperm, reflecting the general use of that term. However, the demonstrated ability to imperfectly fertilize eggs using spermatids emphasizes the more technically accurate use of “spermatogenesis” to refer to the mitotic or meiotic process of germ cell to spermatid conversion (spermatogonia→[mitosis]→primary spermatocytes {diploid}→[meiosis]→secondary spermatocytes {haploid}→[meiosis]→spermatid), and “spermiogenesis” to refer to the process of spermatid to sperm conversion which occurs in the context of close association with Sertoli cells to facilitate natural delivery of the paternal genome.

For steps of the methods disclosed herein for which there are multiple medical technologies available (such as in vitro expansion of stem cells), any one or more of these medical technologies can be used within the spirit of the methods disclosed herein, as can any such medical technologies developed during the lifetime of this patent.

### Overview

In one aspect, the present invention provides methods for creating sperm cells having chromosomes directly derived from an adult female's cells (as opposed to sperm cells for a male that have an X chromosome from his mother).

In one aspect, the present invention provides methods for creating egg cells having chromosomes directly derived from an adult male's cells (as opposed to egg cells for a female that have an X chromosome from her father).

According to one embodiment of this aspect of the invention, a diploid germ cell from a female is provided. The diploid germ cell can be derived using methods and materials known to those of skill in the cloning and developmental biology arts, including, but not limited to, the following methods reviewed herein below: extraction from the ovarian surface epithelium, generation from non-germ stem cells (or other adult cells) which either are retrodifferentiated and then differentiated, by cloning, parthenogenesis or transdifferentiation. Once the Y-chromosome-deficient female germ cell has been derived, spermatogenic capacity is then provided to the female germ cell using one or more of the following techniques: testicular drug delivery, gene therapy vectors, natural Y chromosomes, and/or mammalian artificial chromosomes. The spermatogenically augmented female germ cells so produced are then prepared and transplanted under conditions effective to insure that the female germ cells are imprinted as are male germ cells and X-inactivated where and when necessary. The resulting female spermatogonia are transplanted into a sterilized male testis or artificial testicular environment (e.g., in vitro spermatogenesis) to provide for the production of sperm carrying the chromosomes of the female from whom the female germ cell was derived.

The viability of such methods in humans is consistent with clinical observations of the ability to repopulate germ cells in the immune-privileged testicular environment, the successful procreations of 47,XXY Klinefelter men and women with their one extra sex chromosome, and the germline propagation of artificial (sex) chromosomes.

### Derivation of Diploid Female Germ Cells

Unlike stem cells of lower animal forms such as the medusa which can be retrodifferentiated, mammalian stem cells are less naturally programmable. However, the ability to so reprogram stem cells creates a multiplicity of methods for treating diseases, fighting aging, replenishing cells of organs (including cosmetic uses such as breast augmentation), and enhancing physiological performance (imagine athletes being able to transplant the stem cells of world champion athletes into their muscles).

Since the retrodifferentiated stem cells will later be differentiated to become germ cells, stem cells as uncommitted as possible are desired. There are multiple sources for such cells: obtaining germ cells from the ovarian surface, obtaining stem cells from menstrual blood, preserving fetal stem cells, in vitro retrodifferentiating stem cells to form embryo-like stem cells, and extracting cells from the inner cell mass of a genetically identical embryo derived by cloning or parthenogenesis.

### Ovarian Surface Epithelium Extraction

In some embodiments, female germ cells are provided by extraction from the ovarian surface epithelium (OSE) of an adult woman. It has long been assumed that oogenesis does not occur in adult mammals: that females are born with all of these eggs they will ever possess, with such haploid oocytes having undergone partial meiosis before going into arrest. Thus the need for the methods disclosed herein to create adult female diploid germ cells, using complex techniques such as cloning or stem cell transdifferentiation. Such techniques, however, can be simplified if not eliminated by extraction of such diploid germ cells from the ovarian surface epithelium (OSE) of an adult woman. In 1995, Antonin Bukovsky and others at the University of Kentucky reported on the discovery of such adult female diploid germ cells [BUK95], which further research has confirmed [BUK06], with techniques being developed to culture such cells to derive oocytes in vitro [BUK07]. For example, as described in [BUK07], ovarian tissue with germ cells is collected during diagnostic laparoscopic examination under general anesthesia, with the surface of each ovary gently scraped with laparoscopic scissors, after which the cells are collected by washing branches of the scissors in tissue culture medium. The collected cells can then be cultured in vitro.

Such extracted OSE germ cells can then have their female imprinting patterns adjusted to male imprinting patterns, and any adult oocyte-specific markers can be removed and replaced in vitro with any needed pre-spermatogenesis male germ cell markers. Such cells can then be compensated for any needed Y chromosome spermatogenesis genes, and cultivated in vivo or in vitro to produce sperm. Such cellular modifications can be applied to female stem cells prepared using the following techniques as well.

### Menstrual Blood Cell Extraction

In 2007, two groups reported the extraction of pluripotent stem cells from menstrual blood [MEN07]. Such cells were able to undergo repeated rounds of cell doublings in vitro, and were capable of differentiating into 9 different lineages: cardiomyocytic, respiratory epithelial, neurocytic, myocytic, endothelial, pancreatic, hepatic, adipocytic and osteogenic. As is, or with in vitro embryonic stem cell and/or primordial germ cell induction techniques, such cells can be used with the methods disclosed herein, with the advantage of easy extraction (one company is preparing a kit for women to collect their menstrual blood for stem cell extraction).

### Fetal Cell Preservation

Collection and preservation of fetal stem cells from the umbilical cord or from a pregnant mother's blood is a growing field. Sample approaches include using centrifugation to separate maternal and a variety of fetal cells (U.S. Pat. No. 5,641,622), and/or where the isolated fetal cells are cryopreserved (U.S. Pat. No. 5,192,553). Capturing and saving such cells of a newly born girl eliminates the germ cell creation steps of the methods disclosed herein, at the cost of having to store such cells until the girl becomes an adult.

### Embryonic-like Stem Cell Induction

Cloning to produce embyronic stem cells, disclosed below, requires the use of embryos. Such use can be avoided by creating embryo-like stem cells from adult cells by retrodifferentiation. One method of making a population of mammalian pluripotent embryonic stem cells [HOG97] is by incubating a population of diploid spermatogenic cells in a composition comprising a growth enhancing amount of basic fibroblast growth factor, leukemia inhibitory factor, membrane associated steel factor, and soluble steel factor. In some embodiments of the present invention, these techniques are extended to non-germ stem cells to be applicable to female non-germ stem cells, since the motivation of this aspect of the methods disclosed herein is the lack of adult female diploid germ stem cells. Such extension can be accomplished using methods and materials familiar to those persons having ordinary skill in the art: for example, in 2007, scientists reported converting adult skin cells (fibroblasts) into germline-competent stem cells [SKN07], first achieving so for mice and then for humans.

Recently, a variety of studies have demonstrated development of male germ cells from bone marrow stem cells, with the male germ cells expressing many known germ and spermatogonial stem cell marker [VI.6 and related VI.3]. The conversion was facilitated in part by treatment of the bone cells with retinoic acid, which can be used to treat female bone marrow stem cells. This may be less surprising since the same researchers demonstrated how to convert adult spermatogonial stem cells into embryonic stem-cell-like cells [VI.7], suggesting a greater ability of adult stem cells to be transformed into more pluripotent and totipotent forms. And in 2007, teams of scientists in Japan and at Harvard/MIT reported on converted adult mouse skin cells into embryonic stem cells [VI.11] by adding four genes to the skin cells (with later teams reporting on using different sets of four genes to convert adult human skin cells), techniques which if adaptable to humans, can provide a source of embryonic stem cells for conversion into sperm without the use of cloning. Once a supply of embryonic-like stem cells are prepared, they can be cultured with factors such as the bone morphogenetic proteins (BMPs) to differentiate into primordial germ cells.

Stem cells acquired/prepared through fetal cell preservation or embryonic-like stem cell induction will need to have their imprinting erased and X chromosomes activated (see below).

### Nuclear Transfer Cloning/Parthenogenesis

For adult females whose fetal cells are not available, mammalian cloning techniques can be used to recreate such cells, with the opportunity to obtain germ stem cells that have the requisite cell surface markers that recognize the Sertoli cell surface to which germ cells bind during spermatogenesis. One such marker protein is the mitochondrial capsule selenoprotein encoded by the GPX-4 gene on chromosome 19. Such markers can be induced on the surface of non-germ embryonic stem cells acquired by other methods (such as embryonic-like stem cell induction) by activating the corresponding gene(s).

One approach for cloning is that of nuclear transfer [CAM97] developed at the Roslin Institute, a method for reconstituting an animal embryo by transferring the nucleus from a quiescent donor cell into a suitable recipient cell. The donor cell is quiescent, in that it is caused to exit from the growth and division cycle at G1 and to arrest in the G0 state. Nuclear transfer may take place by cell fusion. One report of higher cloning success rates in mice [WAK98] was by transferring cumulus nuclei from a female ovary into an enucleated female oocyte. The reconstituted embryo then acts as a source of stem cells to be artificially differentiated into primordial germ cells or naturally developed (primordial germ cells are detectable in the epithelium of an extraembryonic membrane, the yolk sac, during weeks one to three after fertilization). While initially certain cells types were preferred for the transfer, recent research is greatly expanding which cells can be used (see, e.g., U.S. Pat. No. 6,011,197).

Primordial germ cells are found in the yolk sac when the embryo is about three weeks old (in humans). Such germ cells migrate by weeks four to five onto the genital ridge, after which they trigger gonad development. Prior to entry into the genital ridge, development of male and female germ cells is indistinguishable. Extraction and expansion of the migrating germ cells at the fourth week provides a source of germ cells (see U.S. Pat. No. 5,744,347) for in vitro cultivation and/or alteration to compensate for the Y chromosome, and can be accomplished by those having ordinary skill in the art. For example, Gearhart et al. [GEA98] report on extracting primordial germ cells 5-9 weeks post-fertilization from gonadal ridges and mesenteries and culturing the germ cells on mouse STO fibroblast feeder layers in the presence of human recombinant leukemia inhibitory factor, human recombinant fibroblast growth factor, and forskolin. Over a 7 to 21 day period, large multicellular colonies developed, for both XX and XY cell cultures. Thomson et al. [THM98] report similar results with blastocyst-derived, pluripotent cell lines, research that is currently being commercialized. The timing of the extraction of the germ cells depends on genetic imprinting constraints discussed below.

Thus, in one embodiment, female diploid germ cells are obtained by cloning an adult woman and isolating and culturing germ cells from the embryo at 7 to 9 weeks post-cloning [VI.1], between the time that the primordial germ cells start migrating out of the yolk sac and the time that the germ cells are colonizing the genital ridge. Such female germ cells will be recognized by persons having ordinary skill in the art as having the benefit of being structurally similar to male germ embryonic germ cells pre-spermatogenesis.

In another embodiment, female diploid germ cells are obtained by cloning an adult woman, isolating embryonic stem cells from the clone, and transforming the stem cells into embryonic germ cells [VI.2], which at least in mice, were cultured in vitro sufficiently competently as haploid pre-sperm cells to allow successful fertilization via ICSI [VI.3, and related VI.6]. The second method has the ethical advantage over the first method by making earlier use of the embryo, while requiring more effort (such as using growth factors) to insure that the germ cells have the proper germ cell markers [VI.4] and imprint erasures. In recent years, many published studies have shown the viability of so converting embryonic stem cells [VI.5]. In another embodiment of this second method the embryonic stem cells are obtained via parthenogenesis, where an egg of an adult woman is chemically or electrically “tricked” into becoming an embryo. While such embryos never result in a successful pregnancy (the male epigenetic information is missing), such parthenogenetic embryos can serve as a source of embryonic stem cells. [KIM07]

### Transdifferentiation

Retrodifferentiation followed by differentiation requires multiple transformations of the original cell.

Transdifferentiation, where stem cells from one family are converted to those of another, might require fewer steps and therefore less risk. The transdifferentiation of mammalian stem cells has been the subject of speculation for decades, with two early articles summarizing this speculation [GOL82] [HAL83]. To date, experiments demonstrating the transdifferentiability of stem cells have been few. One example reported conversion of primordial germ cells into hematopoietic cells in vitro [RCH95], the inverse of which is one pathway needed for the methods disclosed herein. A more recent achievement is that of converting human neural stem cells into blood stem cells [BJO99]. Transdifferentiated cells will need to have their imprinting erased and X chromosomes activated (see below).

Embyonic stem cell induction and cloning, and stem cell transdifferentiation, methods raise the problem of epigenetic reprogramming of the original adult cell, as multiple studies have reported the abnormal phenotypes of clones for the few cases where cloned embryos managed to be born all. However, it is also observed that progeny of cloned animals have normal phenotypes, since their DNA inherited from a cloned parent were normally reprogrammed during gametogenesis in the parent, similar to the reprogramming experienced by the females cells in the methods disclosed herein when injected into the male testicles.

### Stem Cell Expansion

Some of the steps for stem cell retro- (or trans-) differentiation, and/or gene therapy for such cells, have low efficiency rates. Those cells successfully altered are cultivated in vitro to produce larger numbers of candidate cells for other alterations. A growing industry is forming around the need for stem and germ cell expansion, for example a system for selective clonogenic expansion of relatively undifferentiated cells, including, without limitation: (a) a tube containing a plurality of beads of a size which permits a plurality of the undifferentiated cells to grow thereon, the beads bearing on their surfaces a selective binding molecule which binds to a surface antigen present on the relatively undifferentiated cells, wherein the antigen is not present on the surfaces of the relatively differentiated cells; (b) means for continuously providing nutrients to the relatively undifferentiated cells growing on the beads, wherein the nutrients are delivered via a fluid which flows through the tube and past the beads; and (c) means for continuously harvesting relatively undifferentiated cells downstream of the beads (PCT Publication WO 96/36696). Such nutrients include leukemia inhibitory factor, seminiferous growth factor and basic fibroblast growth factor, which have been shown to promote long-term proliferation of primordial germ cells in culture [RES92][NIK97].

More recently has been the achievement of in vitro undifferentiated proliferation of embryonic stem cells, which retained the ability to differentiate into three embryonic germ layers (endoderm, mesoderm and ectoderm) [THM98], as well as earlier work with culturing yolk sac stem cells. Retinoic acid is known to be a meiosis-inhibiting factor, and thus can be used to control whether or not stem cells undergo mitotic or meiotic divisions in vitro while their numbers are being expanded.

Thus, in some embodiments, combinations of the above-described techniques are used to create an adult female diploid germ cell. However, these techniques are insufficient to enable female spermatogenesis. Genes on the male's Y chromosome still have to be accounted for, since diploid female germ cells are similar to germ cells from males that have deleted Y chromosomal fertility loci as seen in some forms of azoospermia. Thus, a trivial transfer of diploid oogonia to a (sterilized) testis would achieve, by itself, dysfunctional sperm unlikely to satisfy regulatory concerns. Thus, as described herein, the present invention further provides for addressing this critical deficiency.

### Y Chromosome Compensation

Studies have shown that genes on the Y chromosome are necessary to promote spermatogenesis, that is, some spermatogenesis genes act autonomously in the germ cells, for example, to control the timing of spermatogenesis. At least three genes/clusters on the distal part of the Y chromosome (Yq) affect spermatogenesis including TSPY [DEL97], DAZ (also referred to as DAZ/SPGY) and RBM/YRRM [MCLA98], in one or more subregions, though only those non-X-homologous genes with near or post-puberty expression in the male germ cell are of interest (e.g., the Sertoli and Leydig cells will continue to express their proteins, since they retain the Y chromosome of the host male whose testes are used to cultivate the female sperm).

In humans, it is recognized that the AZF (AZoospermia Factor) region of the Y chromosome, when altered, leads to fertility problems in males, with genetic deletions leading to little or no sperm in male ejaculates.

Three subregions have been proposed, designated AZFa (azoospermia, Sertoli-cell only phenotype); AZFb (azoospermia, meiotic arrest); and AZFc (azoospermia/oligospermia), with a possible fourth region AFZd (mild oligozoospermia).

For example, one such gene (or gene cluster) for spermatogenesis in humans is DAZ (Deleted in AZoospermia), located in intervals 6E and/or 6D of the distal portion of the long arm of the Y chromosome. The DAZ gene (or cluster, as several copies have been detected in interval 6) is approximately 3.1 kb in size, and encodes a 366 amino acid protein homologous in certain domains to several RNA binding proteins, the DAZ protein restricted to late spermatids and sperm. Alterations (such as deletions) in DAZ typically result in reduced sperm counts. Another gene family is the germ-cell-specific nuclear proteins RBMY (RNA-Binding Motif Y) mostly found in the AZFb subregion, in particular RBMY1 which is active in the nuclei of spermatogonia, primary spermatocytes, and round spermatids [OST01]. Microdeletions in the AFZb region severely reduce spermatogenesis (RBMY is a multiple copy gene), indicating that RBMY homologs on the X chromosomes and autosomes don't compensate for the loss of some copies of the RBMY genes (with the homologs binding differently), a fatal problem for human female germ cells that don't have any RBMY genes (complete deletion, similar to the complete sterility of the rare 46,XX men with a normal male phenotype [ABU01]). RBMY deletion illustrates an important difference between spermatogenesis in humans and mice, since for mouse germ cells, RBMY-deficiency does not lead to sterility but to abnormal sperm development. [SKR07].

Stem cells transplanted from females necessarily lack the needed Y chromosome spermatogenic genes, and thus cannot be developed into normal sperm without the additional intervention provided by the methods of the present invention. Genes such as the RBMY genes, or the products they express, should be provided in the testicular environment along with the female stem cells for female spermatogenesis. For males, after the primary spermatocytes undergo the two meiotic divisions, the resulting spermatids have haploid chromosomes. Half of these spermatids have only the X chromosome and yet continue to develop into mature sperm, due to cytoplasmic bridge connections between X and Y spermatids. Spermatogenic factors are thus needed to help, at least, the initial retrodifferentiated/transdifferentiated stem cell develop to the primary spermatocyte stage.

Thus, in some embodiments, the methods of the present invention include using one or more of the at least four methods known for providing spermatogenic factors: incorporation of (parts of a) natural Y chromosome or of an artificial chromosome with some Y chromosome genes into the female stem cells, testicular drug delivery, gene therapy treatment of the female stem cells, and mixing in tagged male spermatogonia. Non-testicular-originating proteins that contribute to spermatogenesis, such as follicle-stimulating hormone and luteinizing hormone, required by and present in both male and females, need not be compensated for necessarily, although in some embodiments, they are provided by administering promoters such as gonadotropin-releasing hormone stimulatory agonist (see, e.g., PCT Publication WO 97/22625). Also, in some embodiments, the methods of the present invention require adding an X chromosome into male cells, preferably by adding a natural X chromosome.

### Mammalian Natural Y or X Chromosomes

In still another embodiment, compensation of XX germ cells for necessary Y chromosome genes includes preparing a sample of human Y chromosomes by bulk isolation flow cytometry, or micro-cell mediated chromosome transfer (MMCT—further discussed below), to be incorporated into the germ cells. The Y chromosomes can be obtained from the cells of a SRY-negative man or woman (for example, in 2000, German scientists reported on a healthy mother/daughter pair [ROT00] whose genotype was SRY-negative 47,XXY). Using SRY-negative cells is preferable where XY sperm are to be produced (some 47-XXY non-mosaic Klinefelter men produce a small number of XY sperm [GUT97]), creating XXY offspring (saving future generations from having to add a Y chromosome). For producing just X sperm, since the SRY gene has no effect on spermatogenesis, the Y chromosomes can be obtained from cells of any XY male, for example, the Y chromosome of a woman's family member (such as a brother or father). Often in chromosome isolation, fluorescent dyes are used (such as Hoechst†33258, or specially designed polyamides that bind to DNA sequences without requiring harsh denaturing conditions). Any resulting sperm that include such tagged male Y-chromosomes will be fluorescent; and thus easier to sort from sperm with an X chromosome from the female. Similar methods of manipulating X and Y chromosomes for producing clones of animals of the opposite sex of the parent animal are found in U.S. patent application 2002/0174449 [WES02]. One advantage of using all or part of natural chromosomes, as opposed to artificial chromosomes (as described below), is that genes on artificial chromosomes may not be imprinted in the same ways as genes on natural chromosomes.

Other embodiments include using minichromosomes. Minichromosomes are naturally occurring chromosomes that have sections of genes deleted or have been fragmented (U.S. Pat. No. 5,288,625 and U.S. Pat. No. 4,464,472). An alternative to using artificial Y chromosomes based as described below is to use mini-Y-chromosomes created by deleting from existing Y chromosomes those genes that do not contribute to spermatogenesis, for example, using telomere directed chromosome breakage to create a mini-Y-chromosome [HEL96] or a mini-X-chromosome [FAR95]. However doing so provides much less gene sequence control than adding specific genes to an artificial chromosome, or using the whole Y chromosome as with MMCT. Similarly, MMCT is preferred for adding a copy of a male's X chromosome to his cells.

### Mammalian Artificial Chromosomes

In other embodiments, compensation for non-existent spermatogenic genes in female stem cells is accomplished by incorporating a mammalian artificial chromosome (MAC) in the female's germ or stem cells that contain the necessary spermatogenic genes obtained from a healthy male.

A mammalian artificial chromosome is a non-integrating DNA construct that can replicate autonomously and be stably maintained alongside endogenous chromosomes, with a fully functional mammalian centromere, replication initiation sites, and mammalian telomeres. The base DNA is engineered from naturally occurring neutral DNA sequences with no genetic information, and plasmid vectors are used to insert heterologous genes at specific integration sites on the artificial chromosome arms [HAD97, HAD00]. MACs can be incorporated into cells such as germ and stem cells. A good review of multiple methods and protocols for using MACs can be found in “Mammalian Artificial Chromosomes: Methods and Protocols”, edited by Vittorio Sgaramella and Sandro Eridani (Humana Press, 2004), incorporated herein by reference.

Other alternate vector systems are available that accommodate large DNAs. Bacteriophage lambda clones may contain up to 22 kilobases(kb); cosmid clones up to 40 kb. DNAs of this size are routinely introduced in the production of transgenic animals. The large capacity cloning vectors include bacteriophage P1, P1 artificial chromosomes (PACs), cosmids, bacterial artificial chromosomes (BACs), yeast artificial chromosomes (YACs) and mammalian artificial chromosomes. Of these, YACs and MACs have several distinct advantages. First, the maximum insert size that can be contained in YACs and MACs are multiple megabases, a region too large to be cloned intact with traditional bacterial cloning systems. This large insert size permits the study of intact genes, multigenic loci, distant regulatory sequences and higher order genomic structure in the context of their native sequences. Decisions need not be made regarding the regulatory relevance of sequences or distances between genes and control elements, as must be done in the synthesis of mini-transgenes in which sequences must be pared. Second, site-specific mutagenesis can be readily and efficiently performed in vivo with the homologous recombination system of the host cell instead of recombinant DNA technology. Point mutations, deletions, insertions and replacements can easily be introduced into a YAC or MAC without producing unwanted alterations. A P1 can maintain up to 100 kb, much less than the maximum capacity of YACs and MACs. PACs and BACs have cloning capacities up to 350 kb, but performing homologous recombination to introduce mutations has not been demonstrated, making the process of mutagenesis more tedious. The preferred vector for the methods disclosed herein are MACs since YACs don't replicate in mammalian cells. However modified YACs containing human centromeres and telomeres seem to function as MACs [IKE98][OKA98].

The spermatogenesis/AZF regions are a subset of 24 megabases of euchromatin and 30 megabases of heterochromatin in the Y chromosome. The heterochromatin is entirely composed of, and the euchromatin is largely composed of, repeated non-coding DNA sequences. If spermatogenesis can occur with all of the coding and only one third of these non-coding DNA sequences, only about 20 megabases of Y chromosome DNA need to be carried in the artificial chromosome, which is the current carrying capacity of at least one (patented) mammalian artificial chromosome (U.S. Pat. No. 6,743,967). At a minimum, (multiple copies of) the 10 to 20 Y chromosome protein-coding genes with no X chromosome homologue are incorporated into the artificial chromosome (except for SRY).

MACs can be introduced into cells by a variety of methods: electroporation, direct uptake (such as by calcium phosphate precipitation), uptake of isolated chromosomes by lipofection [LOR99], by microcell fusion, microinjection or by lipid-mediated carrier systems. Methods of specific targeted incorporation of heterologous genes into the MAC are available without extraneous random integration into the genome of recipient cells, such as the use of homology targeting vectors. Here, the heterologous gene of interest is subcloned into a targeting vector which contains nucleic acid sequences that are homologous to nucleotides present in the MAC. The vector is then introduced into cells containing the artificial chromosome for specific site-directed integration into the MAC through a recombination event at sites of homology between the vector and the chromosome. The homology targeting vectors may also contain selectable markers for ease of identifying cells that have incorporated the vector into the MAC as well as lethal selection genes that are expressed only upon external integration of the vector into the recipient cell genome.

Two such homology targeting vectors are lambdaCF-7 and p-lambdaCF-7-DTA [HAD97, HAD00], commercialized by the Canadian company Chromos Molecular Systems under the tradename ACE System.

The ACE product line consists of the Platform ACE artificial chromosome, the ACE Targeting Vector for loading genes onto the artificial chromosome, and ACE Integrase, an enzyme for allowing multiple rounds of loading. One Chromos patent (U.S. Pat. No. 6,743,967) claims a capacity of about 20 megabases, which is the size of the Y chromosome with only one-third of the non-coding sequences in the heterochromatin and euchromatin, and the coding sequences of the euchromatin. One advantage of the large size of ACEs is that they can be purified using flow cytometry.

Genes such as SRY and SMCY (which codes for some of the H-Y antigens) are typically are not incorporated into the MAC. Those post-puberty expressed spermatogenic genes on the Y chromosome, such as DAZ and RBMY, are incorporated into the MAC. Promoters to control the timing of their expression (for example using a gene that codes for resistance to a drug, which is administered to the male when the female stem cells are transplanted) can also be incorporated into the artificial chromosome. Such control minimizes any possible abnormalities, since this artificial pseudo-Y chromosome is reminiscent of the cause of Klinefelter's Syndrome (males with an XXY sex chromosome). Harding et al. [HAR98] report on a switching transgene expression using a tetracycline-regulatable system (also claimed in [BUJ99]), while Burcin et al. [BUR99] use the antiprogestin mifepristone. Genes that have been found to be useful for transforming adult somatic cells into embryonic-like stem cells can also be added to the artificial chromosome.

Still other types of mammalian artificial chromosomes can be used in accordance with the invention, such as described in, e.g., U.S. Patent Applications 2007/0004002 and 2003/0064509.

### Incorporating Natural/Artificial Chromosomes into Germ Cells

Once a sample of natural or artificial Y chromosomes (or a combination thereof) has been prepared, and a supply of XX germ cells has been prepared, a variety of in vitro techniques can be used to incorporate the Y chromosomes into the XX germ cells, after which the XXY germ cells can be transplanted into the testicular environment for development into sperm. Such altered cells can be mitotically cultivated in vitro to increase their supply before transplantation. Y chromosomes can be tagged with a reporter gene, such as for a fluorescent protein, to ease the sorting out of any Y sperm produced. U.S. Pat. No. 6,936,469, which is incorporated herein by reference, discloses one way to incorporate the Y chromosomes into germ cells by mixing the Y chromosomes with a cationic lipid/polymer delivery agent (to neutralize the charge of the Y chromosome), and then putting the mixture in contact with the germ cells. Optionally, one can first apply ultrasound or electrical energy to the germ cells to promote the incorporation. The resulting 47,XXY female germ cells are very similar to germ cells of non-mosaic 47,XXY Klinefelter men who can produce sperm, with the male (and thus female equivalents) cells undergoing mitosis and meiosis as is, or with some of the 47,XXY cells undergoing trisomy-rescue during initial stages of mitosis to form 46,XY cells as seen in normal men. Similarly, natural and artificial X chromosomes (and mixtures thereof) can be prepared, and added to male diploid germ cells using these in vitro techniques (with trisomy-rescue induced to convert the 47,XXY male cells to more female-like 46,XX cells).

Micro-cell Mediated Chromosome Transfer. In some embodiments, natural Y chromosomes (with or without the SRY gene) are used instead of artificial Y chromosomes. One way to use natural Y chromosomes is by the process of micro-cell mediated chromosome transfer (MMCT), which has been achieved for cellular engineering purposes involving the Y chromosome [VIJ05]. MMCT is usually a five step process as follows [DOH03]. First, cells from which the Y chromosomes are to be obtained are treated with the drugs colcemid and colchicines, which arrests the cells in the metaphase stage, with prolonged exposure interfering with mitotic spindle formation, and causing the cells to multinucleate and form nuclear membranes around individual or small numbers of chromosomes. Second, the treated cells are centrifuged in the presence of cytochalasin B, which disrupts microfilaments and prevents cells from returning in interphase, leading to micro-cell extrusion. Third, the micro-cells are collected and filtered though membranes with pore sizes as small as 3 micrometers, to select for micro-cells containing a single chromosome. Sorting out of Y-chromosome-only micro-cells can occur at this stage, with the advantage of allowing genes to be added to the sole Y chromosome that have been found useful for transforming adult somatic cells into embryonic-like stem cells. Fourth, these micro-cells are fused to recipient female diploid germ cells, typically by adding phytohemagglutinin-P (PHA-P) to cause cell agglutination and polyethylene glycol (PEG) to dissolve cell membranes. Fifth, fused germ cells with an added Y chromosome are selected, if not selected earlier, (using Y chromosome probes see, e.g., U.S. Pat. No. 5,840,482 [GRA98] and U.S. patent application 2006/0064770 [FRE06]) for further cultivation, epigenetic adjustment and differentiation into sperm cells. A similar five step process can be used for adding an extra X chromosome to male cells. Given the great expense of producing and ensuring the quality of isolated Y and X chromosomes, a (commercial) center can be established to create a bank of isolated Y and X chromosome micro-cells, so that the costs of producing such cells can be distributed across many women and men seeking to use such chromosomes. One advantage of using natural chromosomes, as opposed to artificial chromosomes, to compensate for missing X or Y chromosomes, is that genes on artificial chromosomes may not be imprinted in the same ways as genes on natural chromosomes.

### Testicular Drug Delivery

In other embodiments, testicular drug delivery techniques are used to provide compensation for any required missing Y chromosome genes. For example (see U.S. Pat. No. 4,622,218), testicularly-acting drug species can be site-specifically and sustainedly delivered to the testes (for the methods disclosed herein, the testes of the volunteer host male) by administering a pharmacologically effective amount of the target drug species tethered to a reduced, blood-testis barrier penetrating lipoidal from of a dihydropyridine-pyridium salt-type redox carrier, e.g., 1,4-dihydrotrigonelline. The target drug species can be some of the proteins coded by spermatogenic genes such as DAZ. Recent experiments have shown that glial cell line-derived neurotrophic factor (GDNF) is an important factor in cultivating spermatogonial stem cells in vitro.

### Gene Therapy

In still other embodiments, any missing spermatogenic genes or gene products are compensated for using gene therapy methods. Such methods will be familiar to persons having ordinary kill in the art. For example, U.S. Pat. Nos. 5,695,935, 5,871,920, and 6,020,476 describe methods to extract a gene from a DNA sample from a male and detect if the gene is altered. Such methods enable an unaltered gene from another male, or a portion thereof encoding a functional protein, to be inserted into cells in which the functional protein is expressed and/or secreted to remedy the deficiency caused by the absence of the native gene in male (or female) cells. Similar techniques can be used to compensate for the DAZ and RBMY1 genes by adding them to one of the X chromosomes of the adult female's cells (a rearrangement similar to the naturally occurring Y-X translocation that results in 46,XX infertile males).

### Tagged Male Spermatogonia

In yet other embodiments, the necessary gene products are provided directly instead of by genetic transcription. Since healthy male spermatogonia already have present the necessary spermatogenic genes, they could be used to express proteins that do not remain internal to the spermatogonia. In one particular embodiment, a sample of male spermatogonia is removed from a male testis before sterilization, and tagged, for example, by adding a fluorescent probe such as Hoechst 33342. These tagged spermatogonia are mixed in with the altered female cells, and the entire mass is transplanted back into the testis. Sperm would be produced, some from the female's cells and some from the male's cells. Using conventional cell filtering techniques, those sperm that are fluorescent are be easily identified and removed, leaving only sperm with chromosomes from the female.

### Imprinting/Epigenetics

Imprinting is one type of process involving the epigenome, i.e., modifications to genes that change the functioning of the gene without changing the DNA sequences that comprises the gene. At least three types of epigenetic modifications have been observed: imprinting via DNA methylation of cytosine residues in CpG dinucleotides (forming differentially methylated regions—DMRs), modification of core histones (a “histone code”), and gene silencing due to non-coding RNAs. There are three types of imprinting modifications: 1) in women, the random total inactivation of one X chromosome in each cell (“X-inactivation”); 2) in men and women, random inactivation of some autosomal genes; and 3) in men and women, modification of a small percentage (100 to 200) of one of each of a pair of autosomal genes depending on the person's sex. Sex-specific imprinting is imparted in the germ line, where inherited maternal and paternal imprinting is erased and new imprinting established according to the individual's sex.

Imprinting partly is a process of functional inactivation, with inactive regions of chromatin having altered methylation patterns at the 5-position of cytosine. DNA methyltransferases (such as the DNMT family) are the only known mammalian enzymes that catalyze the formation of 5-methyl cytosine (i.e., adding a methyl group CH3⁻ to a cytosine); and thus play a key role in establishing and maintaining methylation patterns during male spermatogenesis, as seen in measurements of substantial levels of DNA methyltransferase mRNA and protein expression in mitotic types A and B spermatogonia, spermatocytes and round spermatids [JUE94]. This is consistent with the presence of DNA methyltransferase during the frequent cell divisions of spermatogonia, where methylation patterns are maintained on each daughter strand of DNA following semiconservative DNA replication. Such DNA methyltransferases also help maintain methylation patterns during DNA replication for somatic cell divisions.

In some embodiments of the methods disclosed herein, spermatogenic female germ cells formed as described above (e.g., by cloning) have their imprints erased, and are then imprinted as male germ cells. Many imprints are the result of gene site-specific DNA methylation, so that before male imprints can be made for female cells (or female imprints for male cells), the old imprints have to be erased, e.g., actively using DNA demethylases or passively during replication if methylation is interfered with (e.g., to remove any DNMT1 that typically maintains methylation patterns during replication), and/or by using histone-modifying enzymes. Experiments show imprinting to be cell-autonomous, with cells having at least one Y chromosome (for example, from XY or XXY males or sex-reversed XY females) able to acquire male imprints.

Much erasure of the previous gametic imprint occurs by the time of genital ridge colonization, and with a relatively late re-establishment in the gametes [SZA95], [BAR97]. After fertilization, much of the paternally inherited DNA is erased much more quickly then the maternally inherited DNA, and remains erased as the germ cells migrate from the yolk sac to the genital ridge, at which point complete erasure starts, since Onyango et al. report that human embryonic germ cells from the gonadal ridge still have imprints [II.4]. Thus germ cells extracted from a cloned female embryo at the time of genital ridge colonization can be made ready for a male imprinting pattern (and other epigenetic modifications) when transplanted to a male testis to experience spermatogenesis.

The results of Keijean [II.3] suggest that some human male germ cell imprints remain erased until imprinting occurs in adult spermatogenesis. Male germ cells undergo mitosis starting from the fetal stage until death. Male germ cells undergo meiosis primarily post-puberty, with about 80% of the germ cells in the adult testis being in the (post) meiotic stages. Imprinting of the male genome starts during fetal stages of mitosis, with the bulk of germ-cell specific methylation patterns being acquired prior to the type A spermatogonia stage. Any epigenetic mitotic/meiotic modifications to male germ cells in the testes that occur post-puberty (e.g., histone-to-protamine exchange, methylation of the maternally-expressed non-coding RNA gene H19 [SOL98], etc.) will also happen to altered female germ cells when transplanted into a post-puberty testicular environment (or in vitro equivalents). To some extent, male germ line imprinting may be a waste of energy (e.g., for any imprinted genes only active in somatic cells), and may not need to be fully replicated, since paternal imprints in the paternal pronucleus are quickly erased shortly after fertilization at the zygote stage, and experience additional erasure as the embryo undergoes two waves of demethylation. Similarly, Kerjean reports that the imprinted gene MEST/PEG1 remains unimprinted throughout spermatogenesis even until the mature spermatozoa stage.

For male germ cell epigenetic modifications that are fully erased in the embryo by the fetal stage, or by the time of adult spermatogenesis post-puberty, one can erase, in vitro, the imprints of a prepared female diploid germ cell (preparation including the addition of an artificial Y chromosome), so that when the prepared germ cells are transplanted into the testicular environment, the female's chromosomes acquire the necessary male epigenetic modifications naturally. Any imprinting establishment that occurs from the fetal period to pre-puberty can be achieved, for example, using the mitotic steps of procedures for in vitro conversion of male embryonic stem cells into embryonic germ cells to similarly cultivate, in vitro, female diploid germ cells that have been reset epigenetically, before the female germ cells undergo testicular transplantation Chuma and colleagues [CHU05] report on extracting mouse primordial germ cells and transplanting them into a natural testicular environment to produce sperm resulting in normal fertile offspring, supporting the idea that epigenetic modifications in the male germ line during mitotic stages of spermatogenesis pre-puberty do not to be exactly established, if at all, but rather are amenable to in vitro engineering. Kono and colleagues [KON07] report on the successful combination of two mouse eggs to produce healthy offspring by deleting part of the H19 gene in one egg to make it more masculine. While the procedure has a low frequency of success, it suggests much of male epigenetics post-paternal-pronucleus erasure is optional, including epigenetic modifications during the pre-puberty mitotic stages of spermatogenesis, despite the large number of fetal mitotic divisions during which methylation patterns are maintained. Some fetal imprinting, such as for the Oct4 and Nanog genes which promote pluripotency, can be blocked from any expression in altered female germ cells. The work of Chuma et al. and Kono et al. lend support to the preference of testicular injection of (mostly) epigenetically-reset Y-chromosome-compensated female diploid germ cells to achieve sufficient epigenetic modifications for normal fertilization.

### X-inactivation

The largest cluster of imprinted genes is the X chromosome itself. In mammalian females with two X chromosomes (one inherited from a male), one of the X chromosomes is inactivated in all somatic cells, except during some stages of oogenesis. In males, the sole X chromosome is always active, except during the latter- and post-stages of spermatogenesis. At the beginning, mitotic, states of spermatogenesis, both of the X and Y chromosomes are transcriptionally active. It is not until shortly after the zygotene-to-pachytene transition that the X and Y chromosomes are inactivated and compartmentalized into a peripheral nuclear subdomain called the sex- or XY-body [TUR07]. In natural male spermatogenesis, raised levels of imprinting/inactivating DNA methyltransferase present cause X-chromosomes to be inactivated during meiosis, which may prevent the initiation of damaging recombination events, with little to no contribution from the Xist inactivation gene (which when deleted in mice still leads to normal spermatogenesis) [HEA97] [CON98].

In some embodiments, female diploid germ cells, either with an XXY genotype or a trisomy-rescued XY genotype, prepared, for example, by either cloning, stem cell transdifferentiation or ovarian germ cell extraction, have any inactivated X chromosomes reactivated before entry into the mitotic stages of spermatogenesis. Such methods will be understood by persons having ordinary skill in the art. For example, at least in mice, it has been shown that fibroblast stem cells can be reprogrammed to a state highly similar to embryonic stem cells, and that with certain transcription factors (Oct4, Sox2, cMyc, Klf4), any inactivated X chromosomes are reactivated [MAH07]. Any similar combination of transcription factors can be used herein to reactivate X chromosomes in female diploid germ cells. Prepared female XY-germ cells, with activated X-chromosomes, when transplanted into the testes, will experience during meiosis the inactivation of normal male XY-germ cells. Prepared female XXY-germ cells, when transplanted, will also experience the necessary X-inactivation, as do germ cells undergoing spermatogenesis in non-mosaic 47-XXY Kleinfelter men [KLE94] [GUT97] and oogenesis in 47-XXY women [ROT00]. As will be understood by persons having ordinary skill in the art, the fully activated female germ cells will have a complement of somatic histones (i.e., the histone coding of the egg and the histone coding due to replacement of protamines in the sperm with maternally deposited histones soon after fertilization), to then undergo chromatin remodeling during spermatogenesis.

### In Vitro or (Cross Species) In Vivo Testicular Environments

Having prepared the female germ cells, the final step is to allow them to mature into sperm by providing a testicular environment suitable to achieve the desired maturation. It is known that testes are immunologically privileged site for allografts [HED83] due to Sertoli cell secretions (cells which are now being used to produce localized immune suppression to minimize rejection during transplantation (U.S. Pat. No. 5,830,460). Animal experiments have shown that most male-to-female grafts (such as skin) are rejected (due in part to H-Y antigens coded by gene(s) on the Y chromosome), whereas transplants made in other sex combinations (for example female-to-male) more often succeed. Also, immunosuppresive drugs can minimize transplant rejection, along with temporary treatment in the thymus of the male host to induce tolerance. Female stem cells transplanted into the testes can survive the months needed for spermatogenesis without being disrupted by the male's immune system rejecting the transplanted cells.

A first testicular environment for female spermatogenesis is due to Brinster and Zimmerman [BRN93] [BRN99] (incorporated herein by reference), who propose correcting genetic defects in males by extracting stem cells from male testes, which are genetically altered in vitro while the testes are treated to destroy all remaining sperm materials. Corrected stem cells are then transplanted back into the testes, where they undergo spermatogenesis in vivo. This repopulation is patchy and results in restoration of full spermatogenesis only at the sites of repopulation. Fertility is, however, restored. Following transplantation, the donor cells fill the seminiferous tubules and spermatogonial stem cells occupy discrete sites on the epithelial basement membrane. With time the stem cells spread throughout the epithelium to form colonies of proliferating spermatogonia and differentiating spermatogenic cells [DOB99]. Stem cells from a second male can be used, or stem cells from a compatible species.

Since then, such in vivo techniques have replicated by Brinster and others, often revealing the plasticity of the testicular environment: e.g., repopulating with germ cells that were frozen, repopulating the testicles of a mouse with germ cells of a rat, long term (4 months) in vitro cultivation of isolated stem cells before repopulation, and use of retroviruses to deliver genes to isolated stem cells before repopulation. Three areas of injection shown to support such transplantation are the seminiferous tubules, the efferent ducts and the rete testis. For larger testes such as seen in primates, the first two areas are more difficult to access (easier to access in mice), while at least for monkeys (and presumably humans) rete testis injection is more practical. The attraction of this method is that the female germ cells acquire, as naturally as possible, the correct male imprinting patterns, since most if not all of male imprinting occurs during spermatogenesis.

A second testicular environment uses in vitro techniques, for example, by culturing germ cells in vitro by contacting said cells with non-tumorigenic Sertoli, Leydig and peritubular cells [MIL95], or cultivating sperm in vitro in a culture broth augmented with spermatogenesis factors such as GDNF and LIF (for example, U.S. patent application 2006/0265774) [SHI06]. A variety of research efforts are directed towards creating artificial organs (for example, U.S. Pat. No. 5,541,107). Using a combination of these techniques, an in vitro testicular environment could be prepared, eliminating the need for a male's sterilized testis. One example of such an in vitro environment [HUE98] facilitated the meiotic differentiation of rat germinal cells (in cultures of whole cell population from rat seminiferous tubules) from the spermatocyte stage into round spermatids. Such round spermatids can then be used to fertilize an oocyte, with all events during spermiogenesis, such as somatic and testis-specific histones being replaced by protoamines, thus being somewhat optional.

In vivo spermatogenesis is preferred over in vitro spermatogenesis. Testicular transplantation (in vivo) techniques are preferable to in vitro techniques for culturing germ cells into sperm cells, for at least three reasons. First, chromosome imprinting occurs naturally in the testicular environment, as opposed to having to be engineered in vitro. Second, signaling between somatic and germ cells occurs naturally in the testicular environment, as opposed to having to be engineered in vitro. Third, isolated sperm, and/or sperm mostly separated from the other ingredients of semen produced in the testicular environment, have been shown to readily pick up strands of DNA and RNA from their in vitro environments, for example, finding a section of frog DNA in mouse sperm being cultivated. More worryingly, reverse transcriptase in sperm can turn the foreign RNA into DNA, which can then be integrated into the nuclear genome.

Testicular transplantation methods are also preferable so that required modifications to sperm are done naturally (e.g., addition of sperm proteins necessary for fertilization, such as PAWP, and one protein that tags sperm mitochondria for destruction ubiquitin), as opposed to having to engineer the modifications in vitro. Similarly, testicular transplantation methods are preferable so that telomerase-mediated telomere maintenance and elongation occurs naturally during the (pachtene) stages of meiosis. Similarly, testicular transplantation methods are preferable so that any preferential nuclear positioning of sperm chromosomes occurs naturally, for later unpacking of the sperm chromosomes after fertilization.

A third testicular environment is possible by cross-species spermatogenesis, for example, achieving rat spermatogenesis using mouse testes [CLO96]. Germ cells from transgenic rats were transplanted into recipient immunodeficient nude mice previously treated with busulfan. In one animal, a ratio of one rat sperm to 39 mouse sperm subsequently occurred among cells present in the epididymides. Sperm of the two species were normal and distinctive in head and tail morphology (indicating sperm morphology is inherent to the germ cell), though it has yet to be determined if cell surface glycoproteins that the rat sperm acquire in the mouse epididymides will allow fertilization with a rat egg.

Rat spermatogenesis in mouse testis implies contact between rat germ cells and mouse Sertoli cells with cellular junctions, and contacts between the rat germ cells and mouse somatic supporting cells. This is surprising, given that the timing of the differentiation process from stem cell spermatogonia to the mature form, are unique and species specific, and suggests that xenogenic spermatogenesis is likely possible for other species, in particular between the primates [MCL98]. Since rats and mice are believed to have diverged between about 10-11 million years ago, which is twice as long as the separation of men and chimpanzees, then it is likely, for example, for human sperm to be formed in chimpanzee testes (in particular transmitochondrial chimpanzees).

### Male Eggs

In another aspect, the present invention provides methods for making “male eggs”. Since adult males have diploid germ cells in their testes, there is no need to recreate such diploid cells. Similar to the procedures above that isolate Y chromosomes from adult male cells (and then incorporate them into female diploid germ cells), the first step for creating male eggs is to isolate X chromosomes from an adult male, and add the X chromosomes to the adult male's germ cells, for example, using the chromosome addition techniques of U.S. Pat. No. 6,936,469 or similar techniques such as micro-cell mediated chromosome transfer.

Next, the germ cells are treated in vitro to erase male imprinting patterns and sperm cell markers so that the male cells have the characteristics of embryonic germ cells, making them more amenable to being imprinted similarly to female germ cells during oogenesis. The male cells can then be increased in number via in vitro mitotic divisions. Some of the resulting male cells will undergo trisomy-rescue, losing the Y chromosome, resulting in germ cells with a 46,XX genotype, the same genotype found in normal female germ cells. This is important because, at least for mice, the presence of the RBMY gene on the Y chromosome proves mostly lethal for the development of eggs from XY germ cells [BUR93], the opposite of the absence of the RBMY gene on the Y chromosome proving lethal for development of sperm from XY germ cells. Alternatively, RNA interference can be used to block the effects of the RBMY gene during oogenesis.

These treated 46,XX male germ cells are then cultivated in vitro to become eggs, using any of the recently published techniques for in vitro oogenesis (e.g., starting with immature oocytes arrested at the first stage of meiotic division and cultivating them in vitro to then be fertilized [CHA91], [RAO05]). For example, in 2003, U. Penn. researchers reported [HUB03] on the derivation of female oocytes from mouse embryonic stem cells. Techniques for cultivating female eggs in vitro can be used for cultivating male eggs in vitro [EPP88], [SCH88] and [QIN07].

### Applications of the Science of Same-Sex Procreation for Manufacturing Entertainment Products

Co-pending patent application Ser. No. 11/831,402, filed 31 Jul. 2007 (incorporated in its entirety herein by reference), discloses business methods for patenting applications of new science, such as same-sex procreation, for the manufacturing of entertainment products. The science of female sperm and male eggs as disclosed herein provides much structure and functionality to use female sperm and/or male eggs as a plot element when such cells are signified with audio and/or visual signals in an entertainment article of manufacture fixed on a medium. Such signals function to physically transform the human mind.

### Non-Offensiveness of Same-Sex Procreation

MPEP Rule 608 (Eighth Edition, September 2007), citing C.F.R. 1.3, states that “If during the course of examination of a patent application, an examiner notes the use of language that could be deemed offensive to any race, religion, sex, ethnic group or nationality, he or she should object to the use of the language as failing to comply with the Rules of Practice.” As many Americans will find the idea of, let alone the discussion of, same-sex procreation to be religiously offensive (since it implies that phrases such as “Honor your mother and father” are not the words of any omniscient god or gods), disclosed herein are arguments on the relative non-offensiveness of same-sex procreation. First, it is noted that MPEP Rule 608 a) violates Constitutional guarantees of Freedom of Speech, and b) violates Constitutional guarantees of Due Process public notice (the phrase “could be deemed” is unconstitutionally vague, as is the use of “obvious” in 35 U.S.C. 103).

Second, any religious offensiveness of scientific language concerning “female sperm” is contrasted to religious treatment of female sperm and procreation. For example, a passage in the Christian New Testament book, Hebrews 11:11, is often translated from the original Greek as “By faith he [Abraham] received power to generate, even though he was past the norm alage and Sarah herself was sterile . . . ” (from the New American Bible as authorized by the Catholic Church). This is a false translation of a scientific error that appears in the original Greek version of this passage from Hebrews, which in light of Greek grammar states that Sarah has received the power to “kataboleen spermatos”—conceive sperm (women having sperm is a false scientific view developed by ancient Greek philosophers, a false view then borrowed by Greek writers of the New Testament). It is unethical to attribute false science to any god, and just as unethical to deliberately mistranslate the alleged word of a god, in this case with regards to female sperm. If such unethical language abuse is not deemed religiously offensive, then the true scientific language concerning female sperm disclosed herein cannot be religiously offensive, for such scientific language to violate MPEP 608.

The same ethics argument is also offered to counter any enforcement of European Patent Convention Article 53(a), which states in part “European patent shall not be granted in respect of inventions the commercial exploitation of which would be contrary to order public or morality; . . . ” The term “morality” as used in 53(a) is completely vague, and the invention disclosed herein is more moral than any European morality based on religious texts which lie about the invention disclosed herein.

### Other Embodiments

Having thus described at least one illustrative embodiment of the invention, various alterations, modifications, and improvements will readily occur to those skilled in the art. Such alterations, modifications, and improvements are intended to be within the spirit and scope of the invention. Accordingly, the foregoing description is by way of example only and is not intended as limiting. The invention is limited only as defined in the following claims and the equivalents thereto.

### BIBLIOGRAPHY

The following materials are incorporated herein by reference in their entireties and for all purposes.
[ABU01] “XX males without SRY gene and with infertility”, Abusheikha et al., Human Reproduction, v16 (2001), 717
[AFL05] “Human primordial germ cells and embryonic germ cells, and their use in cell therapy”, Aflatoonian and Moore, Current Opinion in Biotechnology, 2005, 16:530-535
[AFL06] “Germ cells from mouse and human embryonic stem cells”, Aflatoonian and Moore, Reproduction, November 2006, 132(5):699-707
[BAR97] “Genomic imprinting in mammals”, Bartolomei and Tilghman, Annual Rev. Genetics, v31 (1997), 493
[BIR02] “Artificial sperm lets lesbians have babies”, The Birmingham Post (England), 19 Jan. 2002
[BJO99] “Turning brain into blood: a hematopoietic fate adopted by adult neural stem cells in vivo”, Bjornson et al., Science, v283 (January 1999), 534
[BRN93] “Repopulation of testicular seminiferous tubules with foreign cells”, Brinster and Zimmermann, PCT WO 93/11228 (June 1993)
[BRN99] “Repopulation of testicular seminiferous tubules with foreign cells, corresponding resultant germ cells, and corresponding resultant animals and progeny”, Brinster, U.S. Pat. No. 5,858,354 (January 1999)
[BUJ99] “Methods for regulating gene expression”, Bujard et al., U.S. Pat. No. 5,888,981 (March 1999), “Methods for producing tetracycline-regulated transgenic mice”, Bujard et al., U.S. Pat. No. 5,922,927 (July 1999)
[BUK95] “Immunohistochemical studies of the adult human ovary: possible contribution of immune and epithelial factors to folliculogenesis”, Bukovsky et al., Am. J Reproductive Immunology, 1995; 33:323-340
[BUK06] “Ovarian germ cells”, Bukovsky et al., Methods in Enzymology, 2006, 419:208-258
[BUK07] “Oocytes derived from ovarian culture initially containing no oocytes”, Bukovsky and Caudle, US. Patent Application 2007/0010013
[BUR93] “Tdy-negative XY, XXY and XYY female mice: breeding data and synaptonemal complex analysis”, Mahadevaiah et al., J Reproduction and Fertility, 1993; 97:151-160
[BUR99] “Adenovirus-mediated regulable target gene expression in vivo”, Burcin et al., Proc. Nat. Acad. Sci., v96 (January 1999), 355,
[CAM97] “Quiescent cell populations for nuclear transfer”, Campbell et al., PCT WO 97/07669 (March 1997)
[CHA91] “Pregnancy after in vitro fertilization of human follicular oocytes collected from nonstimulated cycles, their culture in vitro and their transfer in a donor oocyte program”, Cha et al., Fertility and Sterility, v55 (January 1991), 109
[CHU05] “Spermatogenesis from epiblast and primordial germ cells following transplantation into postnatal mouse testis”, Chuma et al., Development, v132 (2005), 117
[CLO96] “Rat spermatogenesis in mouse testis”, Clouthier et al., Nature, v381 (May 1996), 418
[CON98] “Imprinting mechanism”, Constancia et al., Genome Research, v8 (1998), 881
[DEL97] “A human candidate spermatogenesis gene, RBM1, is conserved and amplified on the marsupial Y chromosome”, Delbridge et al., Nature Genetics, v15 (February 1997), 131
[DOB99] “Computer assisted image analysis to assess colonization of recipient seminiferous tubules by spermatogonial stem cells from transgenic donor mice”, Dobrinski et al., Mol. Reprod. and Devel., v53 (1999), 142
[DOH03] “Microcell-mediated chromosome transfer (MMCT): small cells with huge potential”, Doherty et al., Mammalian Genome. 2003; 14:583-592
[EPP88] “Growth and development of mouse oocytes in vitro”, Eppig et al., in In Vitro Fertilization and Other Assisted Reproduction, Annals of the New York Academy of Science, Vol. 541 (1988), 205-210
[EVA77] “Direct evidence of the capacity of the XY germ cell in the mouse to become an oocyte”, Evans et al., Nature, v267 (1977), 430
[FAR95] “Generation of a human X-derived minichromosome using telomere-associated chromosome fragmentation”, Farr et al., EMBO Journal, v14 (November 1995), 5444
[FRE06] “Methods for identifying a cell or an embryo containing a Y chromosome”, Frendewey et al., US. Patent Application 2006/0064770 (March 2006)
[GEA98] “Derivation of pluripotent stem cells from cultured human primordial germ cells”, Shamblott et al., PNAS, v95 (November 1998), 13726
[GOL82] “Connections between the hematopoietic, nervous and germ-cells systems”, Golub, Nature, v299 (1982), 483
[GOK06] “A prospective on stem cell research”, Gokhale and Andrews, Seminars in Reproductive Medicine, 2006; 24(5):289-297
[GRA98] “Y chromosome specific nucleic acid probe and method for determining the Y chromosome in situ”, Gray and Weier, U.S. Pat. No. 5,840,482 (November 1998)
[GUT97] “Segregation of sex chromosomes into sperm nuclei in a man with 47,XXY Klinefelter's karyotype: a FISH analysis”, Guttenbach et al., Human Genetics, v99 (1997), 474
[HAD97] “Artificial chromosomes, uses thereof, and methods for preparing artificial chromosomes”, Hadlaczky and Szalay, PCT WO97/40183 (October 1997)
[HAD00] “Artificial chromosomes, uses thereof and methods for preparing artificial chromosomes”, Hadlacsky and Szalay, U.S. Pat. No. 6,025,155 (February 2000) and U.S. Pat. No. 6,077,697 (June 2000)
[HAL83] “Stem cell is a stem cell is a stem cell”, Hall, Cell, v33 (May 1983), 11
[HAR98] “Switching transgene expression in the brain using an adenoviral tetracycline-regulatable system”, Harding et al., Nature Biotechnology, v16 (June 1998), 553
[HEA97] “X-Chromosome inactivation in mammals”, Heard et al., Annual Review of Genetics, v31 (1997), 571
[HED83] “Immune privilege in the testis”, Head et al., Transplantation, v36 (1983), 423
[HEL96] “Mini-chromosomes derived from the human Y chromosome by telomere directed chromosome breakage”, Heller et al., PNAS, v93 (July 1996), 7125
[HOG97] “Compositions and methods of making embryonic stem cells”, Hogan et al., PCT WO 97/32033 (September 1997)
[HUB03] “Derivation of oocytes from mouse embryonic stem cells”, Hubner et al., Science, 2003 May 23; 300:1251-1256
[HUE98] “Meiotic differentiation of germinal cells in three week cultures of whole cell population from rat seminiferous tubules”, Hue et al., Biology of Reproduction, v59 (August 1998), 379.
[II.3] Kerjean A., et al. “Establishment of the paternal methylation imprint of the human H19 and MEST/PEGI genes during spermatogenesis”, Hum Mole Gen. 2000; 9(14):2183-7
[I1A] Onyango, et al., “Monoallelic expression and methylation of imprinted genes in human and mouse embryonic germ cell lineages”, PNAS. 2002 Aug. 6; 99(16):10599-10604
[IKE98] “Construction of YAC-based mammalian artificial chromosomes”, Ikeno et al., Nature Biotechnology v16 (May 1998), 431
[JIM98] “Mammalian sex determination: joining pieces of the genetic puzzle”, Jimenez and Burgos, BioEssays, v20 (1998), 696
[JUE94] “Regulation of DNA methyltransferase expression during spermatogenesis”, Kathleen Jue, McGill University Masters Thesis (Nov. 1994)
[KIM07] “Recombination signatures distinguish embryonic stem cells derived by parthenogenesis and somatic cell nuclear transfer”, Kim et al., Cell Stem Cell, v1 (September 2007), 1-7
[KLE94] “Klinefelter's syndrome: new and rapid diagnosis by PCR analysis of XIST gene expression”, Kleinheinz and Schulze, Andrologia. 1994; 26:127-129
[KON07] “High frequency generation of viable mice from engineered bi-maternal mothers”, Kawahara et al., Nature Biotechnology, 2007 September; 25(9):1045-50
[LOR99] “Methods for producing recombinant mammalian cells harboring a yeast artificial chromosome”, Loring et al., U.S. Pat. No. 5,981,175 (November 1999)
[MCL91] “Development of the male gonad”, McLaren, BioEssays, v13 (1991), 151
[MCL95] “Germ cells and germ cell sex”, McLaren, Phil. Trans. R. Soc. Lond. B. v350 (1995), 229
[MCLA98] “Genetic disorders and spermatogenesis”, McLachlan et al., Reprod. Fertil. Dev., v10 (1998), 97
[MEN07] “Endometrial regenerative cells: a novel stem cell population”, Meng et al., J Translation Medicine, v5 (2007), 57
[MIL95] “Conditionally immortalized germ cell lines”, Millan et al., PCT WO 95/02041
[NAY06] “In vitro-differentiated embryonic stem cells give rise to male gametes that can generate offspring mice”, Nayernia K et al., Dev Cell. 2006 July; 11(1):125-132; and “Derivation of male germ cells from bone marrow stem cells”, Nayernia K et al., Lab Invest. 2006 July; 86(7):654-63
[NAY07] “Putative human male germ cells from bone marrow stem cells”, Drusenheimer et al., in “Gamete Biology: Emerging Frontiers in Fertility and Contraceptive Development” (Nottingham University Press, 2007), pages 69-76—paper presented at February 2006 conference
[NIK97] “Leukaemia inhibitory factor stimulates proliferation of prospermatogonial stem cells”, Nikolova et al., Reprod. Fertil. Dev., v9 (1997), 717
[OKA98] “Mammalian artificial chromosomes”, Okazaki et al., PCT WO 98/08964 (March 1998)
[OST01] “Specific localizations of RBM1a in the nuclei of all cell types except elongated spermatids within seminiferous tubules of the human”, Osterlund et al., Int. J of Andrology, 2000; 24:272-277
[QIN07] “Induction of oocyte-like cells from mouse embryonic stem cells by co-culture with ovarian granulose cells”, Qing et al., Differentiation, 2007, 1432-1436 (e-pub before publication)
[RAO05] “In vitro maturation of oocytes”, Rao and Tan, Seminars in Reproductive Medicine, 2005; 23(3):242-247
[RCH95] “Primordial germ cells are capable of producing cells of the hematopoietic system in vitro”, Rich, Blood, v86 (1995), 463
[RES92] “Long term proliferation of mouse primordial germ cells in culture”, Resnick et al., Nature, v359 (October 1992), 550
[ROT00] “An SRY-negative 47,XXY mother and daughter”, Rottger et al., Cytogenet Cell Genet. 2000; 91 (1-4):204-7
[SAS98] “Mouse primary spermatocytes can complete two meiotic divisions within the oocyte cytoplasm”, Sasagawa et al., Biology of Reproduction, v58 (1998), 248
[SCH88] “Factors affecting the developmental capacity of mouse oocytes undergoing maturation in vitro”, Schroeder et al., in In Vitro Fertilization and Other Assisted Reproduction, Annals of the New York Academy of Science, Vol. 541 (1988), 197-204
[SCH03] “Derivation of oocytes from mouse embryonic stem cells”, Hubner et al., Science, 23 May 2003; 300(5623):1251-1256
[SCH05] “Generating oocytes and sperm from embryonic stem cells”, Kehler et al., Seminars in Reproductive Medicine, 2005; v23(3):222-233
[SCH06] “Embryonic stem cells as a potential source of gametes”, Ko et al., Seminars in Reproductive Medicine, 2006; v24(5):322-329
[SHI06] “Method of growing sperm stem cells in vitro, sperm stem cells grown by the method, and medium additive kit to be used in growing sperm stem cells in vitro”, Shinohara and Shinohara, US. Patent Application 2006/0265774 (November 2006)
[SIL97] “Shared genetic motherhood”, Lee Silver, “Remaking Eden” Avon Books (1997), 189-190
[SKN07] “Generation of germline-competent induced pluripotent stem cells”, Okita, et al., Nature, 2007 (advance online publication), doi:10.1038/1nature05934 and “In vitro reprogramming of fibroblasts into a pluripotent ES-cell-like state”, Wernig et al., Nature, 2007 (advance online publication), doi:10.1038/nature05944
[SOL98] “Imprinting”, Solter, Int. J. Dev. Biol, v42 (1998), 951
[SZA95] “Biallelic expression of imprinted genes in the mouse germ line: implications, for erasure, establishment, and mechanisms of genomic imprinting”, Szabo and Mann, Genes & Development, v9 (1995), 1857
[TES05] “Ethics and synthetic gametes”, Bioethics, v19n2, 2005, 146-166; page 157 discusses the difficulties making female sperm
[THM98] “Embryonic stem cell lines derived from human blastocysts”, Thomson et al, Science, v282 (November 1998), 1145
[THO05] “Genomic imprinting and assisted reproductive technology: connections and potential risks”, Thompson and Williams, Seminars in Reproductive Medicine, 2005; 23(3):285-295
[VIJ05] “The human Y chromosome suppresses the tumorigenicity of PC-3, a human prostate cancer cell line, in the athymic nude mice”, Vijayakumar et al., Genes Chromosomes Cancer. 2005 December; 44(4):365-72
[WAK98] “Full term development of mice from enucleated oocytes injected with cumulus cell nuclei”, Wakayama et al, Nature, v394 (July 1998), 369
[WES02] “Method for generating cloned animals using chromosome shuffling”, West and Cibelli, US. Patent Application 2002/0174449 (November 2002)
[VI.1] Turnpenny L et al., “Derivation of human embryonic germ cells: an alternate source of pluripotent stem cells”, Stem Cells 2003; 21:598-609; see also Liu SR et al., “Human embryonic germ cell isolation from early stages of post-implantation embryos”, Cell Tissue Res. 2004; 318:525-531; see also Durcova-Hills G et al., “Pluripotential stem cells derived from migrating primordial germ cells”, Differentiation. 2001; 68:220-226; see also Richards A J et al., “Differentiation of murine premigratory primordial germ cells in culture”, Biology of Reproduction. 1999; 61:1146-1151; see also Nakagawa S et al., “In vitro studies on PGC or PGC-like cells in cultured yolk sac cells and embryonic stem cells of the mouse”, Arch Histol Cytol. 2000; 63(3):229-241; see also Stewart CL et al., “Stem cells from primordial germ cells can reenter the germ line”, Dev Biol. 1994; 161:626-8
[VI.2] Geijsen N, “Derivation of embryonic germ cells and male gametes from embryonic stem cells”, Nature. 2004 Jan. 8; 427(6970):148-154; see also Aflatoonian B and Moore H, “Germ cells from mouse and human embryonic stem cells”, Reproduction. 2006; 132:699-707; see also Toyooka Y et al., “Embryonic stem cells can form germ cells in†vitro”, PNAS, 2003 Sep. 30; 100(20):11457-11462; see also West J and Daley G, “In vitro gametogenesis from embryonic stem cells”, Curr Opin Cell Bio. 2004; 16:688-692
[VI.3] Nayernia K et al., “In vitro-differentiated embryonic stem cells give rise to male gametes that can generate offspring mice”, Dev Cell. 2006 July; 11(1): 125-132
[VI.4] Kee K et al., “Bone morphogenetic proteins induce germ cell differentiation from human embryonic stem cells”, Stem Cells December 2006 December; 15(6):831-7; see also Chen S et al., “Self-renewal of embryonic stem cells by a small molecule”, PNAS. 2006 14 Nov.; 103(46):17266-71
[VI.5] Nagano M, “In vitro gamete derivation from pluripotent stem cells: progress and perspective”, Biol Reprod. 2007 Jan. 10; [Epub]; see also Aflatoonian B and Moore H, “Germ cells from mouse and human embryonic stem cells”, Reproduction. 2006 November; 132(5):699-707; see also Ko K and Scholer H R, “Embryonic stem cells as a potential source of gametes”, Semin Reprod Med. 2006 November; 24(5):322-9; see also Kehler J et al., “Derivation of germ cells from embryonic stem cells”, Ernst Schering Res Found Workshop. 2006; (60):125-42; see also Kehler et al., “Generating oocytes and sperm from embryonic stem cells”, Semin Reprod Med. 2005 August; 23(3):222-33; see also Baughman J M, “In vitro generation of germ cells: new techniques to solve current issues”, Ann N Y Acad Sci. 2005 December; 1061:33-40; see also West J A and Daley G Q, “In vitro gametogenesis from embryonic stem cells”, Curr Opin Cell Biol. 2004 December; 16(6):688-92
[VI.6] Nayernia K et al., “Derivation of male germ cells from bone marrow stem cells”, Lab Invest. 2006 July; 86(7):654-63
[VI.7] Guan K, Nayernia K et al., “Pluripotency of spermatogonial stem cells from adult mouse testis”, Nature. 2006 Apr. 27; 440:1199-1203 [VI.11] Okita K et al., “Generation of germline-competent induced pluripotent stem cells”, Nature. 2007 Jul. 19; 448:313-317 and Wernig M et al., “In vitro reprogramming of fibroblasts into a pluripotent ES-cell-like state”, Nature. 2007 Jul. 19; 448:318-324.

What is claimed:

1. A sperm, comprising a chromosome of human female origin.

2. The sperm of claim 1, wherein said chromosome is male-imprinted.

3. The sperm of claim 2, wherein said sperm is effective to fertilize a human ovum.

4. The sperm of claim 1, further comprising at least one artificial chromosome.

5. The sperm of claim 1, wherein said chromosome is an X-chromosome.

6. The sperm of claim 5, wherein said X-chromosome is male-imprinted.

7. The sperm of claim 6, wherein said sperm is effective to fertilize an ovum.

8. A method for human same-sex reproduction to produce an offspring of two females, comprising introducing a sperm of claim 1 with an ovum under conditions effective to enable said sperm to fertilize said ovum.

9. A method for human female-female same-sex reproduction to produce a female offspring of two human females, comprising introducing a spermatozoon of claim 6 with a human ovum under conditions effective to enable said spermatozoon to fertilize said human ovum.

10. A multimedia entertainment work fixed on a readable medium comprising at least one audio or visual signal that signifies a sperm of claim 1.
